(19) United States
(12) Patent Application Publication
(10) Pub. No.: US 2003/0157031 A1
(43) Pub. Date: Aug. 21, 2003

(54) Method of and apparatus for forming and administering fine particles

(76) Inventors: Lalit Chordia, Pittsburgh, PA (US); Poongunran Muthukumaran, Pittsburgh, PA (US); Brian Moyer, Pittsburgh, PA (US)

Correspondence Address: POONGUNRAN MUTHUKUMARAN, THAR TECHNOLOGIES<INC, 730 WILLIAM PITT WAY, PITTSBURGH, PA, 15238, US

(21) Appl. No.: 10/361,348

(22) Filed: Feb. 6, 2003

Related U.S. Application Data
(60) Provisional application No. 60/355,248, filed on Feb. 8, 2002.

(51) Int. Cl. A61L 9/04; A61K 9/14; B29B 9/00

(52) U.S. Cl. 424/46; 264/5

(57) ABSTRACT

Present invention provides a method and device for the formation of fine particles of a desired substance and for the immediate subsequent administration to an external environment like human or animal tissues. It also provides ways to control the output and solvent, antisolvent concentrations in the particles.

### CROSS-REFERENCE TO RELATED APPLICATION

This application claims benefit from the Provisional patent application Serial No. 60/355,248 and entitled METHOD OF AND APPARATUS FOR FORMING AND ADMINISTERING FINE PARTICLES, teachings of which are incorporated herein by reference.

### BACKGROUND OF THE INVENTION

1. Field of Invention

The present invention relates to a method and device for production and immediate administration of fine particles of desired substances to an external environment. Such external environments include human and animal tissues.

2. Background and Prior Art

The field of drug formulation and delivery has been an intense area of research for quite some time. Traditionally, molecules are delivered by infusion-products, oral formulation or injectables. Even though some drugs are generally water-soluble, they are not delivered by oral means because of sensitivity to enzymes in the digestive system. Poorly water-soluble drugs pose further bioavailability issues that warrant novel formulations. Because the typical means of administration is by injection, there is growing concern over patient compliance, safety and comfort. Healthcare companies are also pushing towards a new method of delivery because of the rising healthcare costs associated with the administration of injectables of such drugs. Additionally, the ability to administer the drug immediately after particle formation may be advantageous in some instances. For example, in a certain application a certain amorphous or crystalline material may be preferred; however, particles may go through a complex set of changes including Oswald Ripening and form larger particles if stored over a period of time, making them unacceptable. Newly-formed particles also have a more highly active surface, which increases bioavailability, and delivering them upon formation reduces the possibility of surface oxidation from storage over a long period of time. Newly-formed particles of the right solid state form, if administered immediately after formation, would have the most bioavailability, making them an ideal choice for certain applications.

Two common methods of delivery, oral and nasal, have been researched extensively. Hallworth et al. (U.S. Pat. Nos. 4,206,758 and 4,353,365) disclose devices in which powdered medicaments are administered to patients via a nozzle either orally or nasally. Newell et al. (U.S. Pat. Nos. 4,627,432 and 4,811,731) also disclose inhalation devices for medicaments that are either in fluid or finely divided solid form. Cook et al. (U.S. Pat. Nos. 4,044,126, 4,414,209 and 4,364,923) disclose the use of aerosol formulations for the delivery of anti-inflammatory steroids and the preparation of the steroid in a crystalline form. The use of supercritical fluids for film and powder formation in general has also been disclosed by Smith (U.S. Pat. No. 4,582,731) and Sievers et al. (U.S. Pat. No. 4,970,093). Sievers et al. (U.S. Pat. No. 5,301,664) then went on to disclose methods and devices in which physiologically active compounds could be administered orally or nasally via dissolving the compound in a supercritical fluids solvent, followed by expansion, thus initiating the formation of a gas-borne dispersion of solute particles.

Building on the previous work of using supercritical fluids to administer drugs to patients, the need still exists for new methods and devices which can deliver drugs in an efficient, safe and comfortable manner. One such example is the formation of fine particles of drugs via supercritical antisolvent precipitation and direct administration to the patient which is disclosed in the present invention.

### SUMMARY OF THE INVENTION

The present invention provides a novel means of formation and subsequent immediate administration of fine particles of a desired substance in the micro- to nanometer range with a narrow size distribution to an external environment. Such an external environment includes but not limited to human and animal tissues. The present invention helps to exploit certain solid state characteristics of desired substances in drug delivery that cannot be exploited with the currently available technologies.

In certain applications, immediate administration of a desired substance after particle formation may be beneficial. For example, in a certain application a certain amorphous or crystalline material may be preferred; however, particles may go through a complex set of changes including Oswald Ripening and form larger particles if stored over a period of time, making them unacceptable. Newly-formed particles also have more highly active surface, which increases bioavailability, and delivering them upon formation reduces the possibility of surface oxidation from storage over a long period of time. Newly-formed particles of the right solid state form, if administered immediately after formation, would have the most bioavailability, making them an ideal choice for certain applications.

### BRIEF DESCRIPTION OF THE DRAWINGS

FIG. 1. Schematic Representation of Apparatus

FIG. 2. Schematic Representation of Apparatus with Additional Energy

FIG. 3. Schematic Representation of Pulmonary Delivery Apparatus

FIG. 4. Schematic Representation of Injection Apparatus

FIG. 5. Schematic Representation of Transdermal Apparatus

FIG. 6. Schematic Representation of Ophthalmic Apparatus

### DETAILED DESCRIPTION OF THE INVENTION

### Definitions:

“Nasal administration” means

Administering the desired substance through the tissues of the nose.

“Pulmonary administration” means

Administering the desired substance through inhalation, respiratory tract tissue and lung tissue.

“Injectable administration” means

Administering the desired substance through injections including subcutaneous, intravenous and intramuscular.

“Transdermal administration” means

Administering the desired substance through skin.

“Ophthalmic administration” means

Administering the desired substance through the tissues of the eyes.

“Nozzle” means

A device to apply or spray the dispersion.

“Polymorph” means

A solid material with a certain crystalline structure.

“Antisolvent” means

A fluid or combination of fluids that does not substantially dissolve the desired substance and reasonably miscible with the solvent.

“Amorphous” means

A solid material with no defined crystalline structure.

### Description

The present invention provides a novel means of formation and subsequent administration of fine particles of a desired substance in the micro- to nanometer range with a narrow size distribution. Utilizing a supercritical antisolvent process, the means involved in the invention can be used for producing micro- and nanoparticles of a desired substance, including, but not limited to, a physiologically active pharmaceutical substance, and for administering particles of the desired substance to an environment external to the administration apparatus. An external environment may include, but not limited to, a target human or animal. Apparatuses for administration to a target human or animal include means of pulmonary, transdermal, ophthalmic, intravenous, subcutaneous, intramuscular, needle or needle-free administration with each having a respective and different external release elements.

In this process, a desired substance is dissolved or dispersed in a solvent that is miscible with a chosen antisolvent. Solvent choice may include, but not limited to, methanol, ethanol, dimethylsulfoxide, tetrahydrofuran, N,N dimethylformamide, toluene, dichloromethane, ethyl ether, heptane, hexane, methylethylketone, methylisobutylketone, acetone, chloroform, fluoroform, carbon tetrachloride, cyclohexane, ethyl acetate, ethyl formate, isbutyl acetate, isopropyl acetate, 2-methyl-1 propanol, pentane, 1-pentanol, 1-propanol, and 2-propanol, ethane, propane, carbon dioxide, nitrous oxide, butane, isobutene, sulfur hexafluoride, or a combination thereof. Antisolvent choice may include, but not limited to, methanol, ethanol, dimethylsulfoxide, tetrahydrofuran, N,N dimethylformamide, toluene, dichloromethane, ethyl ether, heptane, hexane, methylethylketone, methylisobutylketone, acetone, chloroform, fluoroform, carbon tetrachloride, cyclohexane, ethyl acetate, ethyl formate, isbutyl acetate, isopropyl acetate, 2-methyl-1 propanol, pentane, 1-pentanol, 1-propanol, and 2-propanol, ethane, propane, carbon dioxide, nitrous oxide, butane, isobutene, sulfur hexafluoride, or a combination thereof. Next, the dispersion is sprayed into a chamber through a nozzle containing a flowing pressure and temperature manipulated antisolvent. Vibration by piezoelectric or magnetorestrictive means may be used within a precipitation chamber to enhance atomization of the dispersion, mass transfer rate of the antisolvent into the droplet and the solvent out of the droplet. Manipulated antisolvent in the chamber expands the dispersion, dissolves the solvent and precipitates the desired substance in the form of fine particles. Upon full precipitation of a batch of a desired substance, the pressure of the antisolvent and chamber are used to administer the formed fine particles to the external environment.

In certain applications, immediate administration of a desired substance after particle formation may be beneficial because of certain solid state characteristics. For example, in certain applications either an amorphous or certain polymorph material may be preferred. An amorphous material may provide increased bioavailability for poorly water-soluble compounds. A particular polymorph may be preferred due to its favorable dissolution kinetics. However, amorphous particles may go through a complex set of changes including Oswald Ripening and form larger particles or different crystalline structures even during a typical shelf life. The advantages of such solid state characteristics cannot be exploited if the formulation is intended to be stored for a period of time. In supercritical fluid precipitation processes, it is possible to produce amorphous particles or control the polymorphic form of the particles. By carefully optimizing the process to produce the desired polymorph and by administering it immediately, the therapeutic ability of even an unstable polymorph can be exploited. Newly-formed particles also have more highly active surface, which increases bioavailability, and delivering them upon formation reduces the possibility of oxidation or degradation from storage over a long period of time. Newly-formed particles of the right solid state form, if administered immediately after formation, would have the most bioavailability and therefore make them an ideal choice for certain applications.

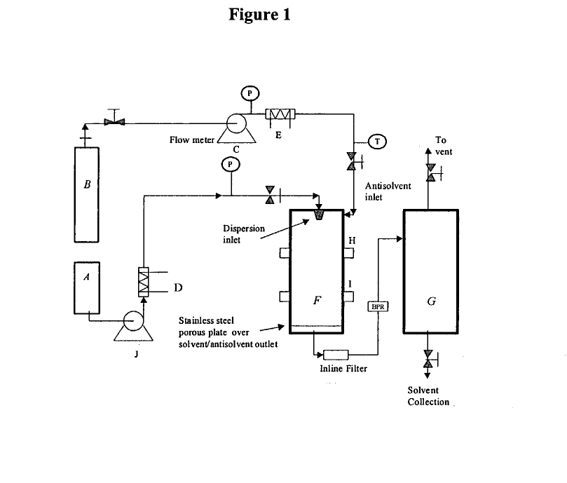

A schematic representation of the apparatus to be used for particle production according to the invention is shown in FIG. 1. Pump C is used to flow the antisolvent at a desired flow rate. The antisolvent stream is pumped through an individual temperature controlled zone E into particle production vessel F. Vessel F is maintained at the desired pressure and desired temperature (near and above the critical point of the antisolvent). The antisolvent inlet is located near the top of the vessel and the antisolvent outlet is located at the bottom of the vessel. Temperature and pressure sensors are employed accordingly at various locations.

The dispersion is sprayed through a nozzle at a desired flow rate. Particles collect in the bottom of particle production chamber F and on a porous material at the antisolvent/solvent outlet, creating a single zone for both precipitation and collection. Solvent exits the chamber through the outlet and enters a back pressure regulator (BPR). Pressure is reduced in the BPR, resulting in solvent and antisolvent separation. Solvent is collected in chamber G. Using the pressure of the antisolvent and chamber F the particles of the desired substance are administered to the external environment through an appropriate external administration element of the respective apparatus.

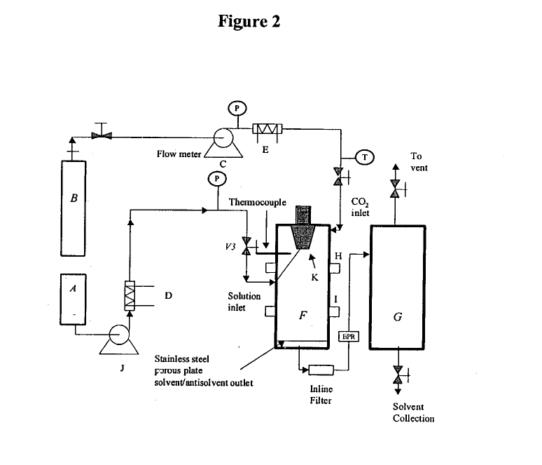

In another embodiment of this invention, a method of adding energy to the process to enhance atomization of the dispersion and mass transfer rate can be practiced. For such embodiment, an apparatus shown in FIG. 2 is to be used.

Pump C is used to flow the antisolvent at a desired flow rate. The antisolvent stream is pumped through an individual temperature controlled zone E into particle production vessel F. Vessel F is maintained at the desired pressure and desired temperature (near and above the critical point of the antisolvent). The antisolvent inlet is located near the top of the vessel and the antisolvent outlet is located at the bottom of the vessel. Temperature and pressure sensors are employed accordingly at various locations.

The dispersion is sprayed through a nozzle at a desired flow rate. Particles collect in the bottom of particle production chamber F and on a porous material at the antisolvent/solvent outlet, creating a single zone for both precipitation and collection. Solvent exits the chamber through the outlet and enters a back pressure regulator (BPR). Pressure is reduced in the BPR, resulting in solvent and antisolvent separation. Solvent is collected in chamber G. Using the pressure of the antisolvent and chamber F the particles of the desired substance are administered to the external environment through an appropriate external administration element of the respective apparatus.

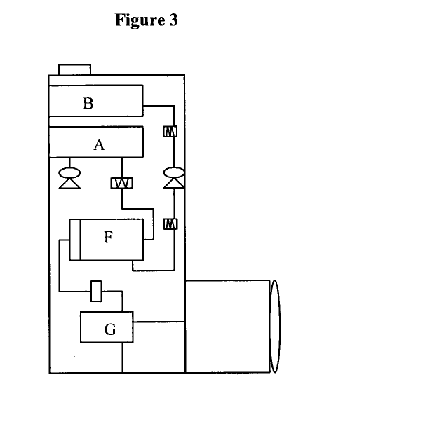

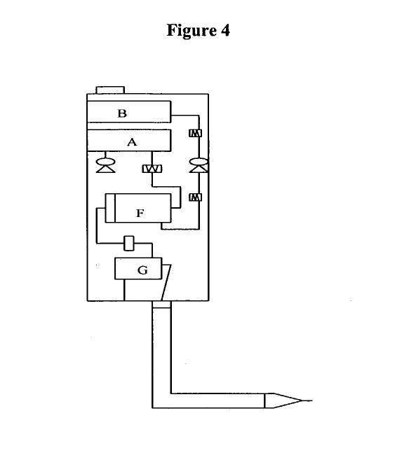

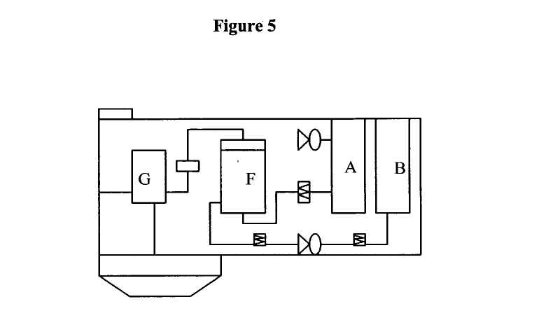

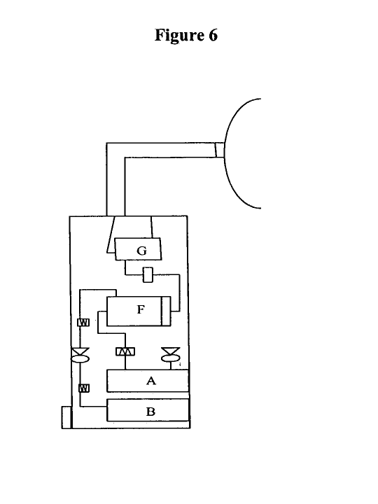

The present invention also includes a means of administering a physiologically active dispersion of fine particles in the antisolvent to target a human or animal tissue immediately after the precipitation as described in the previous paragraphs of the invention. Both formation and administration of fine particles occur in and from a single apparatus. The embodiment of the apparatus may take the form of pulmonary (FIG. 3), injectable (FIG. 4), transdermal (FIG. 5) or ophthalmic (FIG. 6) administration. The methods and processes for particle formation are the same for each route of administration, as shown in FIG. 1 or FIG. 2 and described above; only scale, size and external administration of the apparatus may vary.

The present invention may require the use of pumps for dispersion and antisolvent flow. Solvent and antisolvent used in the invention can be recycled. The present invention provides a means for the control of solvent and antisolvent concentrations in the particles of the desired physiologically active substances. Additionally, the amount of desired substance administered can be controlled. The device of the present invention can have intermittent or continuous flow-of solvent and antisolvent and it can be portable, mobile or stationary.

We claim:

1. A method of formation of a desired substance as fine particles and means of subsequent administration to an external environment comprising:
a. Using the desired substance that is soluble in a solvent but has minimal solubility in an antisolvent.
b. Using an antisolvent that is miscible with a solvent.
c. Applying a dispersion having at least a solvent and the desired substance into a chamber.
d. Applying an antisolvent at or near supercritical conditions to the applied dispersion.
e. Using the pressure of the antisolvent to propel the particles of the desired substance to an external environment.

2. The method as recited in claim 1 including an antisolvent that is physiologically compatible.

3. The method as recited in claim 1 including the addition of energy to the process to enhance atomization of the dispersion and mass transfer rate to produce smaller particles.

4. The method as recited in claim 1 including particle formation and subsequent administration of a desired physiologically active substance.

5. The method as recited in claim 1 including administration to a target human or animal.

6. The method as recited in claim 1 wherein a certain desired substance may require ephemeral solid state characteristics that are lost or relatively unstable if not administered immediately after particle formation or if stored for a long period of time, and having the necessary means to form said characteristics.

7. The method as recited in claim 1 including recycling of antisolvent.

8. The method as recited in claim 1 including recycling of solvent.

9. The method as recited in claim 1 having intermittent or continuous flow of dispersion or antisolvent, or both.

10. An apparatus for the administration of a desired substance following particle formation comprising:
a. Using a desired substance, solvent and antisolvent, which is near or above the critical point, to form fine particles.
b. Applying a dispersion having at least a solvent and the desired substance into a chamber.
c. Applying an antisolvent at near or supercritical conditions to the applied dispersion.
d. Using the pressure of the antisolvent inside the chamber to propel the desired substance to an external environment.

11. The apparatus as recited in claim 10 wherein the administration of the desired substance containing dispersion requires the use of a pump.

12. The apparatus as recited in claim 10 wherein the application of the antisolvent requires the use of a pump.

13. The apparatus as recited in claim 10 including formation of particles of a desired physiologically active substance.

14. The apparatus as recited in claim 10 including recycling of antisolvent.

15. The apparatus as recited in claim 10 including recycling of solvent.

16. The apparatus as recited in claim 10 having intermittent or continuous flow of solvent or antisolvent, or both.

17. The apparatus as recited in claim 10 wherein said apparatus reduces antisolvent concentration in the particles of the desired physiologically active substance.

18. The apparatus as recited in claim 10 wherein said apparatus reduces solvent concentration in the particles of the desired physiologically active substance.

19. The apparatus as recited in claim 10 having a controllable output.

20. The apparatus as recited in claim 10 administering a desired physiologically active substance to a target human or animal.

21. The apparatus as recited in claim 20 wherein particles of a certain desired substance may have ephemeral solid state characteristics that are lost or relatively unstable if not administered immediately after particle formation or if stored for a long period of time, and having the necessary means to immediately administer particles of a desired substance with said characteristics.

22. The apparatus as recited in claim 10 and claim 21, being portable, mobile or stationary.

23. The apparatus as recited in claim 22 wherein said apparatus may be used for pulmonary administration.

24. The apparatus as recited in claim 22 wherein said apparatus may be used for nasal delivery.

25. The apparatus as recited in claim 22 wherein said apparatus may be used with an intravenous formulation for administration through a needle- or needle-free injection.

26. The apparatus as recited in claim 25 having controls to inject the required amount of a desired physiologically active substance, solvent, and antisolvent and maintain the associated pressure and temperature.

27. The apparatus as recited in claim 22 wherein said apparatus may be used for transdermal administration.

28. The apparatus as recited in claim 22 wherein said apparatus may be used for ophthalmic administration.
